(19) United States
(12) Patent Application Publication
(10) Pub. No.: US 2005/0064480 A1
(43) Pub. Date: Mar. 24, 2005

(54) Association of FHOD2 with common type 2 diabetes mellitus

(75) Inventor: Shoulian Dong, San Jose, CA (US)

Correspondence Address: AFFYMETRIX, INC;ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA (US)

(21) Appl. No.: 10/917,647

(22) Filed: Aug. 13, 2004

Related U.S. Application Data
(60) Provisional application No. 60/495,624, filed on Aug. 15, 2003.

(51) Int. Cl. C12Q 1/68; C07H 21/04; C07K 14/72; C07K 16/28

(52) U.S. Cl. 435/6; 435/69.1; 435/320.1; 435/325; 530/350; 530/388.22; 536/23.5

(57) ABSTRACT

FHOD2 has been identified as a type 2 diabetes susceptibility gene. Methods for diagnosing and treating type 2 diabetes and methods for identifying compounds for use in the diagnosis and treatment of diabetes are disclosed. Improved diagnostic methods for early detection of a risk for developing type 2 diabetes mellitus in humans, and screening assays for therapeutic agents useful in the treatment of type 2 diabetes mellitus, by analyzing the FHOD2 gene or gene products from FHOD2, including variants forms of FHOD2, are disclosed. Indicators of diabetes include variant forms of the FHOD2 protein, variant forms of FHOD2 pre-mRNA or mRNA or variant forms of the genomic DNA of the FHOD2 gene or DNA surrounding FHOD2.

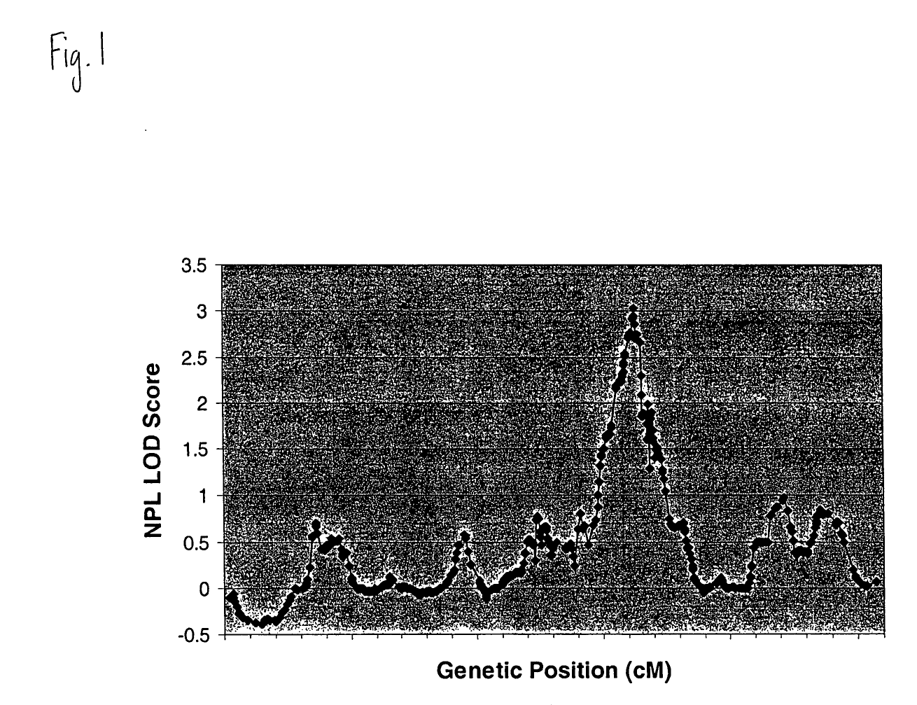

### RELATED APPLICATIONS

This application claims the priority of U.S. Provisional Application No. 60/495,624 filed Aug. 15, 2003, the disclosure of which is incorporated herein by reference in its entirety.

### FIELD OF THE INVENTION

The invention is related to a genomic region that is linked to type 2 diabetes. Methods of identifying patients at risk of developing diabetes or impaired fasting glucose (IFG), or impaired glucose tolerance (IGT) (together termed “impaired glucose homeostasis” [IH]) are disclosed.

### BACKGROUND OF THE INVENTION

Type 2 diabetes is a disease of unknown molecular basis but thought to result from defects in insulin action and insulin secretion. Twin studies indicate that there is a genetic component to the disease but the genetic defects responsible for the majority of cases have not yet been identified. For some relatively uncommon variants of the disease, causative mutations have been identified. For example, mutations have been found in the insulin and insulin-receptor genes, in glucokinase and in the HNF-4α and HNF-1′α transcription factors. The disease is thought to be oligogenic, with two or more genes contributing to the phenotype.

### SUMMARY OF THE INVENTION

A human gene that is linked to type 2 diabetes melittus is disclosed. The gene, FHOD2, also known as FMNL2, may be used as a target to identify drugs to treat type 2 diabetes. The expression of the gene may be used as a method of diagnosing diabetes or of identifying individuals who are at risk for developing diabetes or impaired fasting glucose (IFG), or impaired glucose tolerance (IGT) (together termed “impaired glucose homeostasis” [IH]). Polymorphisms in the FHOD2 gene or in the region surrounding FHOD2 may be used to identify individuals who are at risk for diabetes or IH. A haplotype that is associated with diabetes and includes at least a portion of the FHOD2 gene may be identified and used as an indicator of individuals who are at risk for diabetes.

In some embodiments any 25 contiguous bases of SEQ ID NO 2 or any 25 contiguous bases of the complement to SEQ ID NO 2 is disclosed. In some embodiments any 25 contiguous bases of accession number NT_{—}052905 or its complement are disclosed. In some embodiments probes that are complementary to the FHOD2 gene, mRNA or pre-mRNA are used to detect the FHOD2 gene or gene products. Also incorporated by reference is accession number NT_{—}005403 which includes the sequence of a chromosome 2 genomic contig that contains FHOD2/FMNL2.

Genes in the FHOD2 gene pathway may be identified as indicators of risk for or as diagnostic of type 2 diabetes. The FHOD2 gene may be analyzed at the RNA level as pre mRNA or mRNA. The expression level of the gene may be analyzed. FHOD2 protein levels may be analyzed. An antibody to FHOD2 may be used to detect the FOD 2 protein. Antibodies to variant forms of FHOD2 may be used to detect variant forms of the FHOD2 protein. Variant forms of FHOD2 that are associated with diabetes may be detected to identify individuals at risk for diabetes or IH. Variant forms of the FHOD2 DNA or RNA may be detected to identify individuals at risk for diabetes or IH. Proteins that interact with FHOD2 or variant forms of FHOD2 may be identified. Variant forms of the FHOD2 that associate differentially with FHOD2 associated factors may be identified.

### DETAILED DESCRIPTION

a) General

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W. H. Freeman Pub., New York, N.Y. and Berg et al. (2002) Biochemistry, 5^{th} Ed., W. H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285 (International Publication Number WO 01/58593), which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com.

The present invention also contemplates many uses for polymers attached to solid substrates. These uses include gene expression monitoring, profiling, library screening, genotyping and diagnostics. Gene expression monitoring and profiling methods can be shown in U.S. Pat. Nos. 5,800,992, 6,013,449, 6,020,135, 6,033,860, 6,040,138, 6,177,248 and 6,309,822. Genotyping and uses thereof are shown in U.S. Ser. Nos. 60/319,253, 10/013,598 (U.S. Patent Application Publication 20030036069), and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188,and 5,333,675, and each of which is incorporated herein by reference in their entireties for all purposes. The sample may be amplified on the array. See, for example, U.S. Pat. No. 6,300,070 and U.S. Ser. No. 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No 6,410,276), consensus sequence primed polymerase chain reaction (CP-PCR) (U.S. Pat. No. 4,437,975), arbitrarily primed polymerase chain reaction (AP-PCR) (U.S. Pat. No. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. No. 6,361,947, 6,391,592 and U.S. Ser. Nos. 09/916,135, 09/920,491 (U.S. Patent Application Publication 20030096235), Ser. No. 09/910,292 (U.S. Patent Application Publication 20030082543), and Ser. No. 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y, 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Ser. No. 60/364,731 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Ser. No. 60/364,731 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. Ser. Nos. 10/063,559 (United States Publication No. US20020183936), 60/349,546, 60/376,003, 60/394,574 and 60/403,381.

b) Definitions

The term “admixture” refers to the phenomenon of gene flow between populations resulting from migration. Admixture can create linkage disequilibrium (LD).

The term “allele’ as used herein is any one of a number of alternative forms a given locus (position) on a chromosome. An allele may be used to indicate one form of a polymorphism, for example, a biallelic SNP may have possible alleles A and B. An allele may also be used to indicate a particular combination of alleles of two or more SNPs in a given gene or chromosomal segment. The frequency of an allele in a population is the number of times that specific allele appears divided by the total number of alleles of that locus.

The term “array” as used herein refers to an intentionally created collection of molecules which can be prepared either synthetically or biosynthetically. The molecules in the array can be identical or different from each other. The array can assume a variety of formats, for example, libraries of soluble molecules; libraries of compounds tethered to resin beads, silica chips, or other solid supports.

The term “biomonomer” as used herein refers to a single unit of biopolymer, which can be linked with the same or other biomonomers to form a biopolymer (for example, a single amino acid or nucleotide with two linking groups one or both of which may have removable protecting groups) or a single unit which is not part of a biopolymer. Thus, for example, a nucleotide is a biomonomer within an oligonucleotide biopolymer, and an amino acid is a biomonomer within a protein or peptide biopolymer; avidin, biotin, antibodies, antibody fragments, etc., for example, are also biomonomers.

The term “biopolymer” or sometimes refer by “biological polymer” as used herein is intended to mean repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above.

The term “biopolymer synthesis” as used herein is intended to encompass the synthetic production, both organic and inorganic, of a biopolymer. Related to a bioploymer is a “biomonomer”.

The term “combinatorial synthesis strategy” as used herein refers to a combinatorial synthesis strategy is an ordered strategy for parallel synthesis of diverse polymer sequences by sequential addition of reagents which may be represented by a reactant matrix and a switch matrix, the product of which is a product matrix. A reactant matrix is a l column by m row matrix of the building blocks to be added. The switch matrix is all or a subset of the binary numbers, preferably ordered, between l and m arranged in columns. A “binary strategy” is one in which at least two successive steps illuminate a portion, often half, of a region of interest on the substrate. In a binary synthesis strategy, all possible compounds which can be formed from an ordered set of reactants are formed. In most preferred embodiments, binary synthesis refers to a synthesis strategy which also factors a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion of a substrate may be subjected to a binary scheme. A combinatorial “masking” strategy is a synthesis which uses light or other spatially selective deprotecting or activating agents to remove protecting groups from materials for addition of other materials such as amino acids.

The term “complementary” as used herein refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

The term “effective amount” as used herein refers to an amount sufficient to induce a desired result.

The term “genome” as used herein is all the genetic material in the chromosomes of an organism. DNA derived from the genetic material in the chromosomes of a particular organism is genomic DNA. A genomic library is a collection of clones made from a set of randomly generated overlapping DNA fragments representing the entire genome of an organism.

The term “genotype” as used herein refers to the genetic information an individual carries at one or more positions in the genome. A genotype may refer to the information present at a single polymorphism, for example, a single SNP. For example, if a SNP is biallelic and can be either an A or a C then if an individual is homozygous for A at that position the genotype of the SNP is homozygous A or AA. Genotype may also refer to the information present at a plurality of polymorphic positions.

The term “Hardy-Weinberg equilibrium” (HWE) as used herein refers to the principle that an allele that when homozygous leads to a disorder that prevents the individual from reproducing does not disappear from the population but remains present in a population in the undetectable heterozygous state at a constant allele frequency.

The term “hybridization” as used herein refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.” Hybridizations are usually performed under stringent conditions, for example, at a salt concentration of no more than about 1 M and a temperature of at least 25° C. For example, conditions of 5× SSPE (750 mM NaCl, 50 mM NaPhosphate, 5 mM EDTA, pH 7.4) and a temperature of 25-30° C. are suitable for allele-specific probe hybridizations or conditions of 100 mM MES, 1 M [Na⁺], 20 mM EDTA, 0.01% Tween-20 and a temperature of 30-50° C., preferably at about 45-50° C. Hybridizations may be performed in the presence of agents such as herring sperm DNA at about 0.1 mg/ml, acetylated BSA at about 0.5 mg/ml. As other factors may affect the stringency of hybridization, including base composition and length of the complementary strands, presence of organic solvents and extent of base mismatching, the combination of parameters is more important than the absolute measure of any one alone. Hybridization conditions suitable for microarrays are described in the Gene Expression Technical Manual, 2004 and the GeneChip Mapping Assay Manual, 2004.

The term “hybridization probes” as used herein are oligonucleotides capable of binding in a base-specific manner to a complementary strand of nucleic acid. Such probes include peptide nucleic acids, as described in Nielsen et al., Science 254, 1497-1500 (1991), LNAs, as described in Koshkin et al. Tetrahedron 54:3607-3630, 1998, and U.S. Pat. No. 6,268,490 and other nucleic acid analogs and nucleic acid mimetics.

The term “hybridizing specifically to” as used herein refers to the binding, duplexing, or hybridizing of a molecule only to a particular nucleotide sequence or sequences under stringent conditions when that sequence is present in a complex mixture (for example, total cellular) DNA or RNA.

The term “isolated nucleic acid” as used herein mean an object species invention that is the predominant species present (i.e., on a molar basis it is more abundant than any other individual species in the composition). Preferably, an isolated nucleic acid comprises at least about 50, 80 or 90% (on a molar basis) of all macromolecular species present. Most preferably, the object species is purified to essential homogeneity (contaminant species cannot be detected in the composition by conventional detection methods).

The term “ligand” as used herein refers to a molecule that is recognized by a particular receptor. The agent bound by or reacting with a receptor is called a “ligand,” a term which is definitionally meaningful only in terms of its counterpart receptor. The term “ligand” does not imply any particular molecular size or other structural or compositional feature other than that the substance in question is capable of binding or otherwise interacting with the receptor. Also, a ligand may serve either as the natural ligand to which the receptor binds, or as a functional analogue that may act as an agonist or antagonist. Examples of ligands that can be investigated by this invention include, but are not restricted to, agonists and antagonists for cell membrane receptors, toxins and venoms, viral epitopes, hormones (for example, opiates, steroids, etc.), hormone receptors, peptides, enzymes, enzyme substrates, substrate analogs, transition state analogs, cofactors, drugs, proteins, and antibodies.

The term “linkage analysis” as used herein refers to a method of genetic analysis in which data are collected from affected families, and regions of the genome are identified that co-segregated with the disease in many independent families or over many generations of an extended pedigree. A disease locus may be identified because it lies in a region of the genome that is shared by all affected members of a pedigree.

The term “linkage disequilibrium” or sometimes referred to as “allelic association” as used herein refers to the preferential association of a particular allele or genetic marker with a specific allele, or genetic marker at a nearby chromosomal location more frequently than expected by chance for any particular allele frequency in the population. For example, if locus X has alleles A and B, which occur equally frequently, and linked locus Y has alleles C and D, which occur equally frequently, one would expect the combination AC to occur with a frequency of 0.25. If AC occurs more frequently, then alleles A and C are in linkage disequilibrium. Linkage disequilibrium may result from natural selection of certain combination of alleles or because an allele has been introduced into a population too recently to have reached equilibrium with linked alleles. The genetic interval around a disease locus may be narrowed by detecting disequilibrium between nearby markers and the disease locus. For additional information on linkage disequilibrium see Ardlie et al., Nat. Rev. Gen. 3:299-309, 2002.

The term “lod score” or “LOD” is the log of the odds ratio of the probability of the data occurring under the specific hypothesis relative to the null hypothesis. LOD=log [probability assuming linkage/probability assuming no linkage].

The term “mixed population” or sometimes refer by “complex population” as used herein refers to any sample containing both desired and undesired nucleic acids. As a non-limiting example, a complex population of nucleic acids may be total genomic DNA, total genomic RNA or a combination thereof. Moreover, a complex population of nucleic acids may have been enriched for a given population but include other undesirable populations. For example, a complex population of nucleic acids may be a sample which has been enriched for desired messenger RNA (mRNA) sequences but still includes some undesired ribosomal RNA sequences (rRNA).

The term “monomer” as used herein refers to any member of the set of molecules that can be joined together to form an oligomer or polymer. The set of monomers useful in the present invention includes, but is not restricted to, for the example of (poly)peptide synthesis, the set of L-amino acids, D-amino acids, or synthetic amino acids. As used herein, “monomer” refers to any member of a basis set for synthesis of an oligomer. For example, dimers of L-amino acids form a basis set of 400 “monomers” for synthesis of polypeptides. Different basis sets of monomers may be used at successive steps in the synthesis of a polymer. The term “monomer” also refers to a chemical subunit that can be combined with a different chemical subunit to form a compound larger than either subunit alone.

The term “mRNA” or sometimes refer by “mRNA transcripts” as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

The term “nucleic acid library” or sometimes refer by “array” as used herein refers to an intentionally created collection of nucleic acids which can be prepared either synthetically or biosynthetically and screened for biological activity in a variety of different formats (for example, libraries of soluble molecules; and libraries of oligos tethered to resin beads, silica chips, or other solid supports). Additionally, the term “array” is meant to include those libraries of nucleic acids which can be prepared by spotting nucleic acids of essentially any length (for example, from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

The term “nucleic acids” as used herein may include any polymer or oligomer of pyrimidine and purine bases, preferably cytosine, thymine, and uracil, and adenine and guanine, respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

The term “oligonucleotide” or sometimes refer by “polynucleotide” as used herein refers to a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) which may be isolated from natural sources, recombinantly produced or artificially synthesized and mimetics thereof. A further example of a polynucleotide of the present invention may be peptide nucleic acid (PNA). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

The term “polymorphism” as used herein refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. Single nucleotide polymorphisms (SNPs) are included in polymorphisms.

The term “primer” as used herein refers to a single-stranded oligonucleotide capable of acting as a point of initiation for template-directed DNA synthesis under suitable conditions for example, buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 15 to 30 nucleotides. Short primer molecules generally require cooler temperatures to form sufficiently stable hybrid complexes with the template. A primer need not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

The term “probe” as used herein refers to a surface-immobilized molecule that can be recognized by a particular target. See U.S. Pat. No. 6,582,908 for an example of arrays having all possible combinations of probes with 10, 12, and more bases. Examples of probes that can be investigated by this invention include, but are not restricted to, agonists and antagonists for cell membrane receptors, toxins and venoms, viral epitopes, hormones (for example, opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

The term “receptor” as used herein refers to a molecule that has an affinity for a given ligand. Receptors may be naturally-occurring or manmade molecules. Also, they can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Receptors are sometimes referred to in the art as anti-ligands. As the term receptors is used herein, no difference in meaning is intended. A “Ligand Receptor Pair” is formed when two macromolecules have combined through molecular recognition to form a complex. Other examples of receptors which can be investigated by this invention include but are not restricted to those molecules shown in U.S. Pat. No. 5,143,854, which is hereby incorporated by reference in its entirety.

The term “solid support”, “support”, and “substrate” as used herein are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305 for exemplary substrates.

The term “target” as used herein refers to a molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term targets is used herein, no difference in meaning is intended. A “Probe Target Pair” is formed when two macromolecules have combined through molecular recognition to form a complex.

FMNL2/FHOD2 is Associated with Common Type 2 Diabetes Mellitus

Diabetes mellitus is a high prevalence illness characterized by high blood glucose levels. The chronic hyperglycemia (high glucose level) of diabetes is associated with long-term damage, dysfunction, and failure of various organs, especially the eyes, kidneys, nerves, heart, and blood vessels.

The vast majority of cases of diabetes fall into two broad etiopathogenetic categories. The first category, type 1 or insulin-dependent diabetes mellitus (IDDM), results from an absolute deficiency of insulin due to autoimmunological destruction of the insulin-producing pancreatic β-cells. Another category, type 2 or non-insulin-dependent diabetes mellitus (NIDDM), which accounts for ˜90% of all diabetes cases, is caused by a combination of resistance of insulin action and an inadequate compensatory insulin secretory response.

The genetic defects responsible for the vast majority of cases of type 2 diabetes have not been identified except for a rare, monogenic form of type 2 diabetes-maturity-onset diabetes of the young (MODY). MODY is characterized by non-ketotic diabetes mellitus, an autosomal dominant mode of inheritance, onset typically before 25 years of age and primary defects in pancreatic β-cell dysfunction. Genetically, it results from mutations in any one of the genes encoding for the glycolytic enzyme glucokinase on chromosome 7 and five transcription factors mapped to chromosome 12 and 20. MODY genes are thought to play, at most, a minor role in common type 2 diabetes.

Linkage analysis for families with multiple affected members can identify the genes that predispose the individual to disease. The identified gene may then be used to identify the biochemical or regulatory pathways involved in pathogenesis. Polymorphisms in the gene may be identified to determine which are associated with the disease phenotype and which may confer protection against the disease phenotype. Known polymorphisms in and near the gene may be genotyped to narrow the region of interest and to identify polymorphisms linked to the disease phenotype. The region may be resequenced in affected individuals to identify novel polymorphisms associated with the disease phenotype. The polymorphisms may be analyzed to determine if one or more is causing or contributing to the phenotype.

Microarray based technologies may be used at each step of the analysis. Mapping arrays which have a preselected set of known SNPs distributed approximately evenly across a genome may be used to identify a genomic region that is linked to a disease phenotype. That region may be analyzed by more targeted genotyping approaches. One approach that may be used is to resequence the area from a plurality of individuals. Novel polymorphisms in affected individuals may be identified during the resequencing. Methods of detecting polymorphisms are disclosed, for example, in U.S. Pat. Nos. 5,858,659, 5,925,525, and 6,300,063.

The American Diabetes Association has sponsored a multicenter project, Genetics of NIDDM (GENNID), to collect samples from diabetes patients and their relatives in four American populations. See, Raffel et al., Diabetes Care 19:864 872 (1996). We have carried out linkage studies with more than 10,000 SNP markers among 117 individuals from Hispanic/Latino diabetes families. Multipoint nonparametric linkage analysis was performed with MERLIN (Abcasis et al. Nat. Gen. 30, 97-101, 2002) for affection status of diabetes. A region on chromosome 2q24 centered at 162.346cM was linked to diabetes with LOD=3.05. Additional linkage regions on chromosomes 17 and 3 were also identified. A genomewide linkage study using 389-395 microsatellite markers for these populations (Gelder Ehm et al. Am. J. Hum. Genet. 66, 1871-1881, 2000) failed to identify significant linkage on chromosome 2 for this population. LOD is the log of the odds ratio of the probability of the data occurring under the specific hypothesis relative to the null hypothesis.

The gene at the peak of linkage was identified as formin homology 2 domain containing 2 (FHOD2 also called FMNL2). Formin homology (FH) or Diaphanous-related formin proteins are highly structured proteins and components of RHO family GTPase signaling pathways that affect cytoskeletal organization and induce transcriptional activation of the serum response element (SRE). Eukaryotic FH proteins consist of several conserved domains that are organized in a precise order. From the N terminus, FH proteins contain a loosely conserved FH3 domain, a GTPase binding domain, highly conserved FH1 and FH2 domains, a coiled-coil, and an autoregulatory domain. The C-terminal autoregulatory domain interacts with residues within and/or between the FH3 and the GTPase binding domain and thus mask the conserved FH1 and FH3 domains. This interaction was associated with kinase signaling pathways, actin-binding proteins and microtubules. Activation of the proteins by truncation of the C-terminal stimulated SRE-mediated transcriptional activation.

FHOD2 was predicted as KIAA1902 from cDNA clones (Nagase et al. DNA Res. 8, 179-87, 2001) (GenBank accession number XP_{—}057927) and recently identified and characterized in silico (Katoh and Katoh, Int J Oncol. 22, 1161-8, 2003). It is not well studied compared to another formin homologue protein, formin homolog overexpressed in spleen (FHOS). Besides spleen, FHOS is also highly expressed in skeletal muscle, lung and other tissues. FHOS was recently found to interact with the N-terminal cytoplasmic domain of insulin-responsive aminopeptidase (IRAP) with its C-terminal domain (Mol. Endocrinol. 17, 1216-29, 2003). It may mediate an interaction between glucose transporter isoform type 4 (GLUT4)/IRAP-containing vesicles and cytoskeleton and participate in exocytosis and/or retention of this compartment. Considering the highly conserved structures and domain sequences among FH proteins, FHOD2 may be similar to or share functionalities with FHOS and may play a key role in glucose homeostasis.

KIAA1902 was initially identified and sequences as a cDNA clone in a library generated from human brain. The predicted function based on homology search was in cell signaling and communication as a serine/threonine kinase receptor type 1. It was found to be highly expressed in all brain regions examined in the Nagase et al. study.

Three common variants among the 10K SNPs fell into the FHOD2 region, each with high LOD score, SNP_A-1511102 (TSC1457378, rs2345903) mapped to 5′UTR, SNP_A-1507899 (TSC0061688, rs727327) and SNP_A-1507852 (TSC0061687, rs727326) mapped to intron. The FMNL2 locus is found in the 314,364 base pairs of SEQ ID NO: 3 which is base pairs 3,401,401 to 3,715,764 of a chromosome 2 contig with GenBank accession number NT_{—}005403.

FHOD2 exons are from positions 1 to 252,186475 to 186558, 207271 to 207351, 213553 to 213629, 223272 to 223355, 225415 to 225567, 239668 to 239776, 243420 to 243496, 245488 to 245581, 271871 to 271945, 276027 to 276137, 279383 to 279532, 281623 to 281724, 283378 to 283690, 284041 to 284250, 289970 to 290094, 291057 to 291259, 292831 to 293065, 294188 to 294256, 294357 to 294437, 296540 to 296669, 300959 to 301122, 302105 to 302206, 304490 to 304588, 305323 to 305446, and 312328 to 314364.

The FHOD2 coding sequence is represented by positions 136 to 252,186475 to 186558, 207271 to 207351, 213553 to 213629, 223272 to 223355, 225415 to 225567, 239668 to 239776, 243420 to 243496, 245488 to 245581, 271871 to 271945, 276027 to 276137, 279383 to 279532, 281623 to 281724, 283378 to 283690, 284041 to 284250, 289970 to 290094, 291057 to 291259, 292831 to 293065, 294188 to 294256, 294357 to 294437,296540 to 296669, 300959 to 301122, 302105 to 302206, 304490 to 304588, 305323 to 305446, and 312328 to 312437.

Variants in the region of FHOD2 have been identified and documented in public databases. For example, dbSNP, lists 922 SNPs in the FMNL2 gene (contig positions 3401401 to 3715764) and 5 variants in the genomic region of FMNL2. Table 1 contains a reference identification number for each of the 927 SNPs in the region.

TABLE 1

| | A | B | C | D | E |
|---|---|---|---|---|---|
| 1 | rs2463 | rs2043741 | rs2678295 | rs4254462 | rs4664570 |
| 2 | rs715046 | rs2068886 | rs2678296 | rs4260194 | rs4664573 |
| 3 | rs715047 | rs2078444 | rs2678297 | rs4274556 | rs4664574 |
| 4 | rs727326 | rs2083098 | rs2678298 | rs4289130 | rs4664575 |
| 5 | rs727327 | rs2083099 | rs2678299 | rs4293523 | rs4664576 |
| 6 | rs727601 | rs2118376 | rs2678300 | rs4297832 | rs4664577 |
| 7 | rs727602 | rs2164402 | rs2678301 | rs4311022 | rs4664578 |
| 8 | rs727603 | rs2272535 | rs2678302 | rs4311023 | rs4664579 |
| 9 | rs734540 | rs2272536 | rs2678303 | rs4328591 | rs4664580 |
| 10 | rs734541 | rs2304556 | rs2678304 | rs4331457 | rs4664581 |
| 11 | rs746780 | rs2304557 | rs2678305 | rs4335900 | rs4664582 |
| 12 | rs747012 | rs2304558 | rs2881328 | rs4359594 | rs4664583 |
| 13 | rs751392 | rs2345872 | rs2881335 | rs4368285 | rs4664584 |
| 14 | rs893328 | rs2345873 | rs2881336 | rs4371310 | rs4664585 |
| 15 | rs893329 | rs2345874 | rs2881338 | rs4371312 | rs4664586 |
| 16 | rs893330 | rs2345875 | rs2881339 | rs4396660 | rs4664587 |
| 17 | rs893331 | rs2345902 | rs3047878 | rs4425036 | rs4664588 |
| 18 | rs920244 | rs2345903 | rs3047882 | rs4426480 | rs4664589 |
| 19 | rs934750 | rs2346181 | rs3047921 | rs4442949 | rs4664590 |
| 20 | rs934751 | rs2346182 | rs3047928 | rs4444472 | rs4664591 |
| 21 | rs1023754 | rs2346183 | rs3047933 | rs4471845 | rs4664592 |
| 22 | rs1065266 | rs2346184 | rs3075786 | rs4482430 | rs4664593 |
| 23 | rs1155778 | rs2346185 | rs3080597 | rs4519464 | rs4664594 |
| 24 | rs1155779 | rs2346186 | rs3080598 | rs4520990 | rs4664595 |
| 25 | rs1344152 | rs2346198 | rs3080600 | rs4528717 | rs4664596 |
| 26 | rs1344153 | rs2346199 | rs3080632 | rs4538152 | rs4664597 |
| 27 | rs1370497 | rs2346200 | rs3080633 | rs4544376 | rs4664599 |
| 28 | rs1370499 | rs2346201 | rs3080636 | rs4547483 | rs4664600 |
| 29 | rs1370500 | rs2346202 | rs3215380 | rs4547484 | rs4992311 |
| 30 | rs1370501 | rs2346203 | rs3215381 | rs4613221 | rs5008216 |
| 31 | rs1370502 | rs2346204 | rs3748941 | rs4637059 | rs5835420 |
| 32 | rs1370503 | rs2346205 | rs3762622 | rs4664100 | rs5835421 |
| 33 | rs1370504 | rs2346206 | rs3811568 | rs4664101 | rs5835422 |
| 34 | rs1437679 | rs2346207 | rs3811569 | rs4664102 | rs5835423 |
| 35 | rs1437680 | rs2346208 | rs3811570 | rs4664103 | rs5835424 |
| 36 | rs1437681 | rs2346209 | rs3811571 | rs4664104 | rs5835425 |
| 37 | rs1465818 | rs2346532 | rs3811572 | rs4664105 | rs5835426 |
| 38 | rs1529998 | rs2346533 | rs3811573 | rs4664106 | rs5835427 |
| 39 | rs1545134 | rs2459776 | rs3811574 | rs4664107 | rs5835428 |
| 40 | rs1561267 | rs2577175 | rs3811575 | rs4664108 | rs5835429 |
| 41 | rs1579050 | rs2577176 | rs3811576 | rs4664109 | rs5835430 |
| 42 | rs1812713 | rs2577177 | rs3811577 | rs4664110 | rs5835431 |
| 43 | rs1837105 | rs2577178 | rs3817620 | rs4664111 | rs5835432 |
| 44 | rs1866115 | rs2577179 | rs3841082 | rs4664112 | rs5835433 |
| 45 | rs1866116 | rs2577180 | rs3845678 | rs4664113 | rs5835434 |
| 46 | rs1866117 | rs2577181 | rs3890370 | rs4664114 | rs5835435 |
| 47 | rs1878632 | rs2577182 | rs3901192 | rs4664115 | rs5835436 |
| 48 | rs1965785 | rs2577183 | rs4035882 | rs4664116 | rs5835437 |
| 49 | rs1985975 | rs2577184 | rs4143274 | rs4664117 | rs5835438 |
| 50 | rs2003332 | rs2577185 | rs4146259 | rs4664118 | rs5835439 |
| 51 | rs2003333 | rs2577187 | rs4146903 | rs4664120 | rs5835440 |
| 52 | rs2028168 | rs2678294 | rs4233659 | rs4664569 | rs5835441 |
| 53 | rs6433983 | rs6715576 | rs6742066 | rs7425826 | rs7595822 |
| 54 | rs6433984 | rs6715843 | rs6742846 | rs7426043 | rs7596254 |
| 55 | rs6433985 | rs6716773 | rs6743018 | rs7558989 | rs7596408 |
| 56 | rs6433986 | rs6717982 | rs6744391 | rs7559217 | rs7599894 |
| 57 | rs6433990 | rs6718081 | rs6744977 | rs7561292 | rs7600114 |
| 58 | rs6434003 | rs6718337 | rs6745613 | rs7561791 | rs7600425 |
| 59 | rs6434017 | rs6718584 | rs6745714 | rs7562904 | rs7600568 |
| 60 | rs6434025 | rs6718773 | rs6746190 | rs7563675 | rs7600624 |
| 61 | rs6434028 | rs6720219 | rs6746284 | rs7565316 | rs7601047 |
| 62 | rs6434042 | rs6720532 | rs6746499 | rs7571665 | rs7602505 |
| 63 | rs6434046 | rs6721601 | rs6746538 | rs7571693 | rs7602756 |
| 64 | rs6434059 | rs6721741 | rs6746777 | rs7573833 | rs7602975 |
| 65 | rs6434060 | rs6722740 | rs6746800 | rs7573890 | rs7604573 |
| 66 | rs6434061 | rs6724305 | rs6747312 | rs7574348 | rs7605220 |
| 67 | rs6434062 | rs6724909 | rs6747765 | rs7574699 | rs7606289 |
| 68 | rs6434068 | rs6725060 | rs6748954 | rs7575177 | rs7606397 |
| 69 | rs6434078 | rs6725656 | rs6748966 | rs7575441 | rs7606592 |
| 70 | rs6434081 | rs6725661 | rs6749146 | rs7576442 | rs7607120 |
| 71 | rs6434082 | rs6725789 | rs6750956 | rs7576568 | rs7607429 |
| 72 | rs6434097 | rs6725954 | rs6751094 | rs7576847 | rs7607506 |
| 73 | rs6434098 | rs6727037 | rs6751151 | rs7579097 | rs7607747 |
| 74 | rs6434099 | rs6727781 | rs6751576 | rs7579243 | rs7608137 |
| 75 | rs6434113 | rs6728118 | rs6751778 | rs7579511 | rs7608463 |
| 76 | rs6434114 | rs6729565 | rs6751924 | rs7579527 | rs7608585 |
| 77 | rs6434115 | rs6729611 | rs6754501 | rs7580023 | rs7609122 |
| 78 | rs6434128 | rs6729957 | rs6754723 | rs7581544 | rs9288090 |
| 79 | rs6434129 | rs6730004 | rs6755173 | rs7581918 | rs9288103 |
| 80 | rs6704965 | rs6730982 | rs6756108 | rs7582423 | rs9288107 |
| 81 | rs6705427 | rs6731407 | rs6756332 | rs7582905 | rs9288108 |
| 82 | rs6705986 | rs6731516 | rs6756367 | rs7582984 | rs9677373 |
| 83 | rs6706132 | rs6731699 | rs6756465 | rs7583872 | rs9677380 |
| 84 | rs6706231 | rs6731738 | rs6757207 | rs7584000 | rs9678076 |
| 85 | rs6706394 | rs6732842 | rs6757219 | rs7585911 | rs9678127 |
| 86 | rs6706407 | rs6733002 | rs6757782 | rs7586195 | rs9679683 |
| 87 | rs6706416 | rs6733442 | rs6757784 | rs7586834 | rs9752336 |
| 88 | rs6707033 | rs6734194 | rs6757877 | rs7587450 | rs9753431 |
| 89 | rs6707730 | rs6734434 | rs6757879 | rs7587658 | rs9967683 |
| 90 | rs6708443 | rs6734534 | rs6757884 | rs7587794 | rs9967814 |
| 91 | rs6708667 | rs6736003 | rs6759772 | rs7588008 | rs9967819 |
| 92 | rs6709494 | rs6736639 | rs6759858 | rs7589218 | rs9989874 |
| 93 | rs6710417 | rs6736856 | rs6760139 | rs7590079 | rs10048775 |
| 94 | rs6710651 | rs6737006 | rs6760242 | rs7590207 | rs10165830 |
| 95 | rs6710689 | rs6737699 | rs6760321 | rs7591466 | rs10166430 |
| 96 | rs6710999 | rs6738344 | rs6760818 | rs7591606 | rs10166703 |
| 97 | rs6712716 | rs6739300 | rs6761178 | rs7591723 | rs10168692 |
| 98 | rs6712717 | rs6739525 | rs6761708 | rs7591868 | rs10168793 |
| 99 | rs6713348 | rs6739557 | rs6761815 | rs7592127 | rs10169491 |
| 100 | rs6713711 | rs6739954 | rs7368679 | rs7592283 | rs10169514 |
| 101 | rs6713836 | rs6740909 | rs7371728 | rs7593411 | rs10171204 |
| 102 | rs6714213 | rs6741131 | rs7421103 | rs7593505 | rs10172230 |
| 103 | rs6714218 | rs6741664 | rs7421149 | rs7593580 | rs10173398 |
| 104 | rs6715038 | rs6741728 | rs7424540 | rs7594969 | rs10173899 |
| 105 | rs10174137 | rs10497111 | rs11326775 | rs11883839 | rs12474369 |
| 106 | rs10177081 | rs10524595 | rs11342086 | rs11884490 | rs12474927 |
| 107 | rs10178618 | rs10538950 | rs11343380 | rs11885624 | rs12474974 |
| 108 | rs10179385 | rs10539703 | rs11344436 | rs11886407 | rs12476186 |
| 109 | rs10179391 | rs10539716 | rs11345920 | rs11891914 | rs12476261 |
| 110 | rs10179418 | rs10554817 | rs11349892 | rs11892222 | rs12478426 |
| 111 | rs10180234 | rs10559962 | rs11360283 | rs11892232 | rs12478681 |
| 112 | rs10180542 | rs10567754 | rs11369015 | rs11892322 | rs12612608 |
| 113 | rs10182254 | rs10580348 | rs11384938 | rs11892659 | rs12613219 |
| 114 | rs10182858 | rs10581091 | rs11389713 | rs11892662 | rs12613900 |
| 115 | rs10182993 | rs10581805 | rs11390817 | rs11892859 | rs12613901 |
| 116 | rs10183530 | rs10584642 | rs11392993 | rs11892925 | rs12613902 |
| 117 | rs10184210 | rs10597937 | rs11403468 | rs11893683 | rs12614414 |
| 118 | rs10184459 | rs10600592 | rs11407117 | rs11897348 | rs12614465 |
| 119 | rs10185455 | rs10605671 | rs11408358 | rs11897616 | rs12614608 |
| 120 | rs10186650 | rs10605918 | rs11408981 | rs11897929 | rs12616141 |
| 121 | rs10188611 | rs10617862 | rs11428785 | rs11898429 | rs12616382 |
| 122 | rs10191051 | rs10624832 | rs11428942 | rs11898643 | rs12616778 |
| 123 | rs10191361 | rs10630545 | rs11430178 | rs11899045 | rs12616906 |
| 124 | rs10193104 | rs10645049 | rs11436361 | rs11899068 | rs12617686 |
| 125 | rs10193625 | rs10646636 | rs11438121 | rs11899286 | rs12618643 |
| 126 | rs10194603 | rs10649381 | rs11440524 | rs11901239 | rs12618892 |
| 127 | rs10195380 | rs10670761 | rs11446146 | rs11901392 | rs12619061 |
| 128 | rs10195727 | rs10672047 | rs11452085 | rs11901762 | rs12619679 |
| 129 | rs10195938 | rs10685899 | rs11455357 | rs11902693 | rs12619706 |
| 130 | rs10200831 | rs10688923 | rs11462086 | rs11904088 | rs12621431 |
| 131 | rs10203197 | rs10694393 | rs11538551 | rs11904617 | rs12621715 |
| 132 | rs10204038 | rs10699311 | rs11674872 | rs12052225 | rs12622247 |
| 133 | rs10206228 | rs10702331 | rs11675841 | rs12052587 | rs12622731 |
| 134 | rs10207275 | rs10717513 | rs11676208 | rs12052694 | rs12623070 |
| 135 | rs10208744 | rs10717805 | rs11676897 | rs12052809 | rs12623225 |
| 136 | rs10208776 | rs10719527 | rs11678890 | rs12104452 | rs12693359 |
| 137 | rs10209069 | rs10803964 | rs11679491 | rs12105467 | rs12693362 |
| 138 | rs10209763 | rs10931054 | rs11680639 | rs12105483 | rs12693378 |
| 139 | rs10210776 | rs10931062 | rs11682487 | rs12151546 | rs12693393 |
| 140 | rs10211164 | rs10931067 | rs11684450 | rs12151547 | rs12693404 |
| 141 | rs10432492 | rs10931068 | rs11684983 | rs12151851 | rs12693405 |
| 142 | rs10432493 | rs10931069 | rs11686482 | rs12185697 | rs12693406 |
| 143 | rs10432494 | rs10931075 | rs11687067 | rs12464681 | rs12693407 |
| 144 | rs10439306 | rs10931079 | rs11687886 | rs12465320 | rs12693415 |
| 145 | rs10460316 | rs10931089 | rs11688690 | rs12466536 | rs12693429 |
| 146 | rs10497100 | rs10931146 | rs11689419 | rs12467610 | rs12986786 |
| 147 | rs10497101 | rs10931150 | rs11692343 | rs12467702 | rs12987041 |
| 148 | rs10497102 | rs10931154 | rs11692786 | rs12468231 | rs12987348 |
| 149 | rs10497103 | rs10931163 | rs11694392 | rs12468606 | rs12987493 |
| 150 | rs10497104 | rs10931189 | rs11694512 | rs12468749 | rs12987733 |
| 151 | rs10497105 | rs10931191 | rs11694575 | rs12468789 | rs12987825 |
| 152 | rs10497106 | rs11267395 | rs11694760 | rs12471183 | rs12988475 |
| 153 | rs10497107 | rs11275578 | rs11695776 | rs12471256 | rs12989510 |
| 154 | rs10497108 | rs11292635 | rs11696004 | rs12471268 | rs12989726 |
| 155 | rs10497109 | rs11306212 | rs11883762 | rs12471699 | rs12989840 |
| 156 | rs10497110 | rs11325472 | rs11883821 | rs12471988 | rs12989943 |
| 157 | rs12990922 | rs13023975 | rs13408985 | rs13021931 | rs13403239 |
| 158 | rs12990935 | rs13024308 | rs13409788 | rs13022419 | rs13404703 |
| 159 | rs12991039 | rs13024504 | rs13409795 | rs13022422 | rs13404820 |
| 160 | rs12991203 | rs13024533 | rs13409883 | rs13023506 | rs13405110 |
| 161 | rs12991474 | rs13026023 | rs13409884 | rs13023529 | rs13405341 |
| 162 | rs12992255 | rs13026560 | rs13409889 | rs13023659 | rs13405868 |
| 163 | rs12992391 | rs13026712 | rs13409892 | rs13023779 | rs13406875 |
| 164 | rs12992900 | rs13026858 | rs13412508 | rs13023928 | rs13406882 |
| 165 | rs12993239 | rs13027160 | rs13412574 | rs13023943 | rs13406999 |
| 166 | rs12993871 | rs13027356 | rs13413144 | | |
| 167 | rs12994519 | rs13028185 | rs13414252 | | |
| 168 | rs12995460 | rs13028472 | rs13414680 | | |
| 169 | rs12998222 | rs13028480 | rs13414887 | | |
| 170 | rs12999189 | rs13028846 | rs13415463 | | |
| 171 | rs12999503 | rs13029234 | rs13415525 | | |
| 172 | rs13000076 | rs13029407 | rs13417781 | | |
| 173 | rs13000092 | rs13029667 | rs13418521 | | |
| 174 | rs13000852 | rs13029771 | rs13418748 | | |
| 175 | rs13001913 | rs13031546 | rs13418821 | | |
| 176 | rs13002771 | rs13033172 | rs13418832 | | |
| 177 | rs13003413 | rs13033209 | rs13418936 | | |
| 178 | rs13003885 | rs13033468 | rs13419000 | | |
| 179 | rs13004473 | rs13034458 | rs13419948 | | |
| 180 | rs13004494 | rs13034520 | rs13421428 | | |
| 181 | rs13005838 | rs13034665 | rs13423608 | | |
| 182 | rs13006032 | rs13382410 | rs13423731 | | |
| 183 | rs13006671 | rs13386403 | rs13424038 | | |
| 184 | rs13007956 | rs13387295 | rs13425532 | | |
| 185 | rs13009223 | rs13387298 | rs13425748 | | |
| 186 | rs13009580 | rs13387427 | rs13426403 | | |
| 187 | rs13010632 | rs13387542 | rs13426471 | | |
| 188 | rs13011152 | rs13388095 | rs13427076 | | |
| 189 | rs13011684 | rs13390698 | rs13427289 | | |
| 190 | rs13011850 | rs13391023 | rs13427485 | | |
| 191 | rs13014071 | rs13396439 | rs13427489 | | |
| 192 | rs13015675 | rs13396834 | rs13427942 | | |
| 193 | rs13016534 | rs13397100 | rs13427990 | | |
| 194 | rs13017355 | rs13397890 | rs13428569 | | |
| 195 | rs13017492 | rs13398440 | rs13429157 | | |
| 196 | rs13018337 | rs13400908 | rs13430331 | | |
| 197 | rs13018434 | rs13401202 | rs13430448 | | |
| 198 | rs13018870 | rs13402243 | rs13430837 | | |
| 199 | rs13020285 | rs13403205 | rs13432002 | | |

Each of these SNPs is either in an intronic region of an untranslated region. Methods to identify polymorphisms in the genomic region containing FHOD2 that are associated with a risk for type 2 diabetes or IH are disclosed. Polymorphisms that are associated with a decreased risk of type 2 diabetes may also be identified. Methods of identifying polymorphisms are well known in the art. In one embodiment the genomic region is resequenced in a plurality of individuals.

This work represents the first to associate FHOD2 with common type 2 diabetes. The results indicate that FHOD2 may be involved in the transcription control of glucose response elements and in some embodiments FHOD2 is used as a target for drugs to treat diabetes and insulin resistance. In some embodiments variations in or around this gene may be associated with the severity and progression of impaired glucose homeostasis. In some embodiments variations in or near this gene may be used to diagnosis diabetes or insulin resistance. In some embodiments variations in or near this gene may be used to predict patient outcome or to predict treatment outcome. In some embodiments variations in or near this gene may be used in one or more pharmacogenetic tests.

FHOD2 may be used in methods to identify compounds useful in the treatment of diabetes and related diseases, methods to determine the predisposition of individuals to diabetes and methods for diagnosis and prognosis of diabetes.

In some embodiments one or more probes to the FHOD2 gene are disclosed. The probes may be for example, oligonucleotides that may be, for example, 20, 25, 30, 50, 60 or 100 bases. The probes are complementary to a region of the FHOD2 gene. The probes may be complementary to either strand of the double stranded DNA. The probes may be complementary to one or more forms of the FHOD2 mRNA. The probes may be complementary to an intron of the FHOD2 gene or to the 5′UTR or 3′UTR of FHOD2. In a preferred embodiment one or more probes that are complementary to a polymorphic form of FHOD2 that is associated with a metabolic disease are disclosed. Probe pairs that include a probe to a wild type form and a probe to a mutant form may be used.

In one embodiment a SNP that is indicative of a haplotype that is associated with a metabolic disorder, for example with type 2 diabetes are disclosed. Haplotypes are described in Gabriel et al., Science 296: 2225-9 (2002), Daly et al. Nat Genet. 29: 229-32 (2001) and Rioux et al. Nat Genet. 29:223-8 (2001), which are each incorporated herein in their entireties by reference for all purposes.

In one embodiment probes that are complementary to the FHOD2 gene in a polymorphic region are disclosed. The probes may be used to genotype polymorphisms in or near FHOD2. Allele specific probes may be used so that one probe hybridizes specifically to one allele of a biallelic polymorphism and another probe hybridizes specifically to a second allele of the biallelic polymorphism. In one embodiment, a plurality of probes wherein at least one probe is complementary to one allele of a biallelic polymorphism in the FHOD2 gene or the region surrounding the FHOD2 gene, within 1000, 2000, 3000 or 5000 bases of the FHOD2 gene, and at least one probe is complementary to a second allele is disclosed. The presence or absence of one or more allele may be detected. The presence of a specific allele of a polymorphism in the region may be associated with diabetes.

In one embodiment a kit for genotyping at least one polymorphism in FHOD2 is disclosed. The kit may comprise a probe that hybridizes to a polymorphic region of the FHOD2 gene, including as non limiting examples, the coding region, the 5′UTR, the 3′UTR, introns and upstream or downstream regulatory regions. The kit may comprise a probe that hybridizes to the FHOD2 gene immediately adjacent to a polymorphism. The probe may further comprise a tag sequence.

Methods of genotyping a polymorphism are known to those of skill in the art and any method of genotyping may be used to determine which alleles are present at one or more polymorphic positions in and around the FHOD2 gene. Genotyping methods may involve methods such as oligo ligation assay (OLA), single base extension (SBE), allele specific hybridization, sequencing, and mass spectroscopy. For additional methods of genotyping see Syvanen, A-C, Nat. Rev. Genet. 2:930-942 (2001), Jenkins and Gibson, Comp. Funct. Genom. 3:57-66 (2002), and Twyman and Primrose, Pharmacogenomics, 4:67-79 (2003), each of which is incorporated herein by reference in its entirety for all purposes. Kits comprising oligonucleotides for the genotyping of polymorphisms in the FHOD2 gene and surrounding region are contemplated herein.

In some embodiments the kit comprises an array. The array may comprise probes for genotyping one or more polymorphisms in the FHOD2 gene and surrounding DNA. The array may also comprise probes for genotyping other polymorphisms in other genes that are predicted to be susceptibility genes for diabetes or for other metabolic disorders. In one embodiment an assay comprising oligonucleotides for a plurality of polymorphisms that are indicative of susceptibility for diabetes is disclosed.

For a discussion of genotyping analysis methods see, for example, Elena and Lenski Nature Reviews, Genetics 4:457-469 (2003), Hirschorn et al. Genetics in Medicine 4:45-61 (2002) and Glazier et al. Science 298:2345-2349 (2002) each of which is incorporated herein by references for all purposes. One method of allele specific genotyping that may be used to genotype selected SNPs in the FHOD2 region is described in Hardenbol et al. Nat Biotechnol. 2003 Jun; 21(6):673-8. Epub May 5, 2003 and in U.S. Patent Publication No. 20040101835. The molecular inversion probe (MIP) assay described in Hardenbol et al. may be used to genotype large numbers of SNPs. Multiplex analysis of more than 1,000, 5,000 or 10,000 probes in a single tube may be performed using the assay. A single probe is used per marker or SNP. The genotypes may be read out using a tag array such as the Affymetrix GenFlex or Tag 3 array. Molecular inversion probes may be designed for a selected subset of SNPs, for example MIPs can be designed and synthesized for more than 50, 100, or 500 of the SNPs in Table 1. Novel SNPs identified by resequencing may be targeted by MIPs and the MIP assay. Smaller numbers of SNPs in the FHOD2 region may be genotyped by any method known in the art.

Polymorphisms in the FHOD2 gene may be used to stratifyhuman patients according to risk of developing type 2 diabetes or metabolic disorders, as such methods relate to indicators of risk for diabetes associated with the FHOD2 gene and polymorphic variants in the FHOD2 gene and in the surrounding region are also disclosed.

### Conclusion

The present inventions provide isolated nucleic acid sequences, probes and methods for identifying mutations that are associated with an increased risk of type 2 diabetes. It is to be understood that the above description is intended to be illustrative and not restrictive. Many variations of the invention will be apparent to those of skill in the art upon reviewing the above description. The scope of the invention should, therefore, be determined not with reference to the above description, but should instead be determined with reference to the appended claims, along with the full scope of equivalents to which such claims are entitled.

1. An isolated nucleic acid comprising a polymorphic variant of SEQ ID NO: 2 wherein the polymorphic variant is associated with a metabolic disorder.

2. The isolated nucleic acid of claim 1 wherein said polymorphic variant is a single nucleotide polymorphism.

3. The isolated nucleic acid sequence of claim 1 wherein the metabolic disorder is altered glucose homeostasis.

4. The isolated nucleic acid sequence of claim 1 wherein the metabolic disorder is type 2 diabetes.

5. A protein encoded by the isolated nucleic acid of claim 1.

6. The protein of claim 5 wherein the polymorphic variant is associated with increased risk of a type 2 diabetes.

7. The protein of claim 5 wherein the polymorphic variant is associated with increased altered glucose homeostasis.

8. An antibody to the protein of claim 5 wherein the protein varies at one amino acid.

9. An antibody to the protein of claim 5 wherein the protein is a truncated form of the protein or wherein the protein has a deletion of up to 10, 20, 30, 50 or 100 amino acids.

10. An antibody to the protein of claim 5 wherein the protein varies at two or more amino acids.

11. An oligonucleotide probe comprising 20 to 100 contiguous nucleotides of a polymorphic variant of SEQ ID NO. 3 or its complement, wherein the probe is complementary to a region comprising the polymorphism and is complementary to a polymorphic variant that is associated with a metabolic disorder.

12. The oligonucleotide probe of claim 11 wherein the metabolic disorder is type 2 diabetes.

13. The oligonucleotide probe of claim 12 wherein the probe is 20 to 50 nucleotides in length.

14. An oligonucleotide probe comprising 20 to 100 contiguous nucleotides of a polymorphic variant of SEQ ID NO. 2 or its complement, wherein the probe is complementary to a region comprising the polymorphism and is complementary to a polymorphic variant that is associated with a metabolic disorder.

15. The oligonucleotide probe of claim 14 wherein the metabolic disorder is type 2 diabetes.

16. The oligonucleotide probe of claim 14 wherein the probe is 20 to 50 nucleotides in length.

17. The oligonucleotide probe of claim 11 wherein the polymorphism is a haplotype tag SNP that is indicative of the presence of a haplotype that is associated with type 2 diabetes.

18. A method of determining if a patient is at increase risk of developing type 2 diabetes comprising:
identifying a risk allele of a polymorphic variant of SEQ ID NO 2 or SEQ ID NO 3 that is associated with an increased risk of developing type 2 diabetes;
determining if the risk allele is present in the patient; and
determining that the patient is at increase risk of developing type 2 diabetes if the risk allele is present.

19. The method of claim 18 wherein said identifying step comprises:
resequencing at least 100,000 bases of SEQ ID NO 3 in a plurality of individuals that have type 2 diabetes;
comparing the sequences obtained to a reference sequence from a healthy individual; and
identifying at least one sequence variant that is present in at least one individual that has type 2 diabetes and absent in the reference sequence, wherein the sequence variant is indicative of a risk allele.

20. The method of claim 19 wherein at least 300,000 bases of SEQ ID NO 3 is resequenced.
